(19) United States
(12) Patent Application Publication
(10) Pub. No.: US 2025/0095818 A1
(43) Pub. Date: Mar. 20, 2025

(54) Method for predicting response to cancer immunotherapy

(71) Applicant: Evaxion Biotech A/S, Hørsholm (DK)

(72) Inventors: Andreas Holm Mattsson, Hørsholm (DK); Christian Garde, Hørsholm (DK)

(21) Appl. No.: 18/576,818

(22) PCT Filed: Jul. 7, 2022
(86) PCT No.: PCT/EP2022/068877

§ 371 Date: Jan. 5, 2024

(30) Foreign Application Priority Data
Jul. 7, 2021 (EP) 21184280.2

(51) Int. Cl. G16H 20/17; G01N 33/68; G16H 50/70

(52) U.S. Cl. CPC G16H 20/17; G01N 33/6857; G16H 50/70; G01N 2333/70539; G01N 2800/52

(57) ABSTRACT

A method for predicting patient responses to cancer immunotherapy, in particular treatment with immune checkpoint inhibitors and/or treatment involving induction of specific antitumour immunity. Also provided are methods of treatment and determination of eligibility for treatment.

### FIELD OF THE INVENTION

The present invention relates to the field of cancer immunotherapy and to a method of determining in advance the per patient most likely outcome of such cancer immunotherapy.

### BACKGROUND OF THE INVENTION

In recent years, an increasing focus has been put on immunotherapeutic approaches when treating patients suffering from cancer (malignant neoplasms). In particular, immune checkpoint inhibitors have shown great promise and also various technologies for targeting cancer-associated or cancer-specific antigens via active immunotherapeutic approaches are employed clinically and in trials.

However, a constant challenge is the fact that not all patients respond satisfactorily to these types of treatments. This has the consequence that a substantial number of cancer patients are subjected to these (quite expensive) types of treatment to no avail—in turn this has economic implications, but it also means that some cancer patients are not offered the treatment that would be their own best option.

Numerous attempts have been made to identify biomarkers that can be used to stratify patient populations to allow medical practitioners to prescribe the optimum treatment to each patient. However, hitherto it has not been possible to identify those patients that will truly benefit from cancer immunotherapeutic approaches to allow highly rational treatment choices among those patients that superficially all appear to be equally suited for the same treatment.

### OBJECT OF THE INVENTION

It is an object of embodiments of the invention to provide methods for identifying cancer patients that will benefit from cancer immunotherapy. Further objects are to provide rational treatment regimens for cancer patients and to provide means for rational decision making in cancer treatment scenarios.

### SUMMARY OF THE INVENTION

It has been found by the present inventor(s) that an integrated measure of the expression of MHC Class I and II isotypes in the tumour microenvironment is of high prognostic value for the outcome of cancer immunotherapy, in particular in cases where the cancer immunotherapy entails administration of immune checkpoint inhibitor agent(s) or where the cancer immunotherapy entails induction of active specific immunotherapy. As evident from the examples, patients who prior to or early into a cancer immunotherapeutic treatment regimen exhibit a low combined expression of MHC Class I and II are far less likely to respond to the therapy than those patients whose combined expression is at a higher level. It turns out that these two subsets of patients form very distinct and readily separable groups. A simple linear multivariate regression model based on historical cancer patient data where the independent variables in the model are expression levels in the tumour microenvironment of MHC Class I and II isotypes and where the dependent variable is an indication of clinical outcome e.g. disease progression, overall survival or response to therapy for each patient in the historical data, and where the data relate to patients who have received a cancer immunotherapy, is highly predictive for the outcome of the same cancer immunotherapy in a patient suffering from the same cancer. So by feeding the patient's HLA expression levels to the model, a clear indication of future treatment success with cancer immunotherapy is provided.

So, in a first aspect the present invention relates to a method for determining whether a human patient suffering from a malignant neoplasm has a low chance of responding to cancer immunotherapy, the method comprising,
a) quantitively determining the expression levels of MHC Class I and II molecule isotypes by the cells of the microenvironment of the patient's malignant neoplasm (“the tumour microenvironment”),
b) calculating a combined expression score value from weighted expression levels of at least HLA-A, -B, -C, -DR isotypes determined in step a, wherein the weights for all of isotypes HLA-A, -B, -C, -DR have the same sign (either positive or negative), and
c) determining that the human patient has a low chance of responding to therapy if the calculated combined expression score value from step b is either
   1) closer to the combined expression score values determined in other patients suffering from the same type of malignant neoplasm and lacking response to therapy after receiving said cancer immunotherapy than to the combined expression score values from other patients suffering from the same malignant neoplasm and respond to said cancer immunotherapy, and/or
   2) not significantly different at a 95% confidence level from the combined expression score values obtained from other patients suffering from the same malignant neoplasm and having exhibited lack of response to therapy after receiving said cancer immunotherapy.

In more simple words, the 1^{st} aspect of the invention provides for a simple method for determining, based solely or at least on measurements of the above-mentioned MHC isotypes, whether a patient diagnosed with a cancer that potentially can be treated with cancer immunotherapy will be likely to benefit from the immunotherapy or not. So the method is in its simplest form a method for predicting the outcome of a cancer immunotherapy in a patient by evaluating—against equivalent historical data—an integrated measure of the expression level in the microenvironment of the patient's malignant cells of the above-mentioned MHC isotypes: if this integrated measure aligns better with the same integrated measure in historical data from patients that respond to therapy than from patients with do not respond, the conclusion is that the patient has a high chance of responding to said immunotherapy.

In related 2^{nd} and 3^{rd} aspects the invention further relates to a method of treatment via cancer immunotherapy and a method for determining patient eligibility for cancer immunotherapy. Thus, the 2^{nd} aspect relates to method for treatment of a human patient suffering from a malignant neoplasm, comprising determining, by the method according to the first aspect of the invention and any embodiments thereof disclosed herein, whether the human patient has a high risk of progression of disease or not responding to therapy if receiving said cancer immunotherapy, and subsequently subjecting the human patient to the cancer immunotherapy if it is determined that the likelihood of responding to therapy Is different from low, and subjecting the patient to palliative or alternative treatment regimens if it is determined that the likelihood of response is low. And, the 3^{rd} aspect relates to a method for determining whether a human patient suffering from a malignant neoplasm is eligible for a cancer immunotherapy, the method comprising determining, by the method according to the first aspect of the invention and any embodiments thereof, the likelihood of the human patient to respond to said cancer immunotherapy and concluding that the patient is eligible for said cancer immunotherapy if the determination reveals that the patient does not have a low likelihood of responding to therapy.

### LEGENDS TO THE FIGURE

FIG. 1: Graph showing Principal Component Analysis of expression levels of the HLA genes.

A: Data from EVX-01 trial with results from 12 enrolled patients.

B: Data from EVX-02 trial with results from 6 enrolled patients.

C: Data from EVX-01 trial with results from 18 enrolled patients.

D: Data from EVX-02 trial with results from 14 enrolled patients.

FIG. 2: Graph showing results of a leave-one-patient-out cross validation study on predictive models generated using LDA (linear discriminant analysis) with PCA (principal component analysis) feature encoding of biomarker expression values. Data are obtained from the EVX-01 clinical trial.

A: Data from EVX-01 trial with results from 12 enrolled patients.

B: Data from EVX-01 trial with results from 18 enrolled patients.

FIG. 3: Graphs showing the responder and non-responder distributions of expression values for the respective HLA genes in the EVX-01 and EVX-02 trials.

A: EVX-01 trial with results from 12 enrolled patients.

B: EVX-02 trial with results from 6 enrolled patients.

C: EVX-01 trial with results from 18 enrolled patients.

D: EVX-02 trial with results from 14 enrolled patients.

FIG. 4: Graph showing prediction results against the days of progression free follow up time (measured in days).

FIG. 5: Graph showing impact on sample source on predictive value of MHC expression analysis.

### DETAILED DISCLOSURE OF THE INVENTION

### Definitions

A “malignant neoplasm” (also termed a “malignant tumour” or “cancer”) is a grouping of abnormal cells that are characterized by excessive cell divisions and growth and have the capacity of growing invasively into healthy tissue and to metastasize. Malignant neoplasms can be solid or—as is the case with leukemia—liquid. In solid tumours, not only the abnormal cells constitute the malignant tissue, since de facto normal cells, such as those of the vascular system and stromal cells of connective tissue, can be present in the malignant tissue. Thus, a malignant neoplasm is characterized by the presence of malignant cells, which make up a substantial part of the affected tissue.

A “tumour antigen” is in the present context an antigen which appears in tumour tissue. Such a tumour antigen can be a tumour-associated antigen or a tumour-specific antigen but also a tumour-tissue associated antigen.

A “tumour-associated antigen” is an antigen, which preferentially appears in cancer and only in low amounts in non-cancer tissue. Numerous examples are known from the literature, cf. below. A non-limiting list of tumour-associated antigens is presented here: HER2/neu, MUC1 (CA15-3), MART1 (Melan A), CEA, gp100, gp75, MAGE1, MAGE2, MAGE3, MAGE13, PRAME (preferentially expressed Antigen of Melanoma), TLP (tumour liberated particles) NY-ESO-1, CA 125, CA72-4, CA 19.9, 5T4, p53 (wild type), Tyrosinase (TRP-1, TRP-2), TOPO2α (topoisomerase II alpha), BAGE1, GAGE1, EGFR, GD-2, GD-3, GM-2, Endostatin, Lipophilin B, HSP90, IGFBP2 (insulin like growth factor binding protein), Cyclin B1, Fibulin, CD20, Cyclin D1, Cathepsin D, AFP, LAGE-1, PAP, PSMA, hCG-β, 1GFBP2, GA733 (or mEGP in mice), HOXA7, HOXB7, HSP-27, HSP-90, GIPC-1, Ep-CAM, TAG-72, S100A7, MC1R, WT-1, Mammaglobin-A, livin, survivin, sTn, globo-H (MBr1), and SSX.

A “tumour-specific antigen” is an antigen, which only appears in cancer cells-typically such antigens are products of random somatic point mutations. These antigens are unique for patient's tumour. “Neoantigens” belong to this category. These antigens can be expression products of genes that characterize a cancer cell. For instance, certain expression products of alternative splice variants of the telomerase gene are considered to be tumour specific because they never have been observed in non-cancer tissue.

A “tumour-tissue associated antigen” is an antigen, which appears in stroma of cancer tissue. The cells in the stroma are not malignant per se but often appear to play a role in tumour development by stimulating the tumour cells and/or by protecting them from the body's normal defence mechanisms and/or by providing angiogenesis enabling tumour growth.

An “immune checkpoint inhibitor” is a substance (typically an antibody) which targets an Immune checkpoint, i.e. targets a regulator of the immune system, which is crucial for self-tolerance but also for cancer cell survival in those cases where malignant cells stimulate the immune checkpoint. The most important immune checkpoints that can be targeted are CTLA-4, PD-1, PD-L1

An “antibody” is in the present context an immunoglobulin of any origin and type. The term thus includes IgA, IgD, IgE, IgG and IgM, but also other antibody formats, e.g. antibodies derived from other species than human beings or antibody fragments or synthetic/semisynthetic antibody analogues, where such antibody fragments or synthetic/semisynthetic antibody analogues are characterized by including—as a minimum—the antigen binding region of an antibody. Further, an antibody may be polyclonal (i.e. non-identical antibodies that share the feature or specifically binding to a particular antigen), or monoclonal, i.e. antibodies that are derived from one B-lymphocyte clone. In the event the antibody is fragment or synthetic or semisynthetic analogue it is typically selected from any of the known formats such as Fab, Fab′, F(ab)₂, F(ab′)₂, F(ab)₂, Fv (typically the VL and VH domains of a single arm of an antibody), single-chain Fv (scFv), dsFv, Fd fragments (typically the VH and CHI domain), and dAb (typically a VH domain) fragments; VH, VL, VhH, and V-NAR domains; minibodies, diabodies, triabodies, tetrabodies, and kappa bodies (see, e.g., Ill et al., Protein Eng 1997; 10:949-57).

“Specific binding” refers to binding between an antigen and an antibody's antigen binding site, where said binding has a higher affinity than what can be observed when allowing the same antibody to interact with a random panel of irrelevant antigens.

The term “immunogenic” refers to the ability of an antigen to elicit a specific adaptive primary or secondary Immune response against said antigen.

The term “immunological adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Microenvironment” of a malignant neoplasm—also termed “tumour micro-environment” herein, is the local composition of extracellular matrix together with malignant and non-malignant cells within a malignant tumour.

### Specific Embodiments of the Invention

### 1^{st} Aspect of the Invention

This aspect of the invention relates to a method for determining whether a human patient suffering from a malignant neoplasm has a low likelihood of responding a later received cancer immunotherapy, the method comprising,
a) quantitively determining the expression levels of MHC Class I and II molecule isotypes by the cells of the microenvironment of the patient's malignant neoplasm,
b) calculating a combined expression score value from weighted expression levels of at least HLA-A, -8, -C, -DR isotypes determined in step a, wherein the weights for all of isotypes HLA-A, -B, C, -DR have the same sign (either positive or negative), and
c) determining that the human patient has a low likelihood of responding to immunotherapy if the calculated combined expression score value from step b is either
   1) closer to the combined expression score values determined in other patients suffering from the same malignant neoplasm and that do not respond to said cancer immunotherapy than to the combined expression score values from other patients suffering from the same malignant neoplasm and having exhibited response to said cancer immunotherapy, and/or
   2) not significantly different at a 95% confidence level from the combined expression score values obtained from other patients suffering from the same malignant neoplasm and having exhibited lack of response to said cancer immunotherapy.

It will be understood that step b in some embodiments can be integrated with the determination in step c: if the values from step a are e.g fed to a neural network or other machine learning model, which has been trained with data sets comprising MHC isotype expression levels aligned with data of clinical outcome after therapy e.g. disease progression, overall survival or response to therapy, no visible calculation as in b will be presented-nevertheless, based on the findings herein, such a machine learning model would nevertheless put particular weight on the HLA isotypes set forth above.

However, it will be understood that the first aspect of the invention therefore also relates to a method for determining whether a human patient suffering from a malignant neoplasm has low likelihood of responding to a cancer immunotherapy, the method comprising,
x) quantitively determining the expression levels of MHC Class I and II molecule isotypes by the cells in the environment of the patient's malignant neoplasm,
y) inputting at least the expression levels of HLA-A, -B, -C, -DR isotypes determined in step a into a neural network or other machine learning model, which has been trained with data sets obtained from cancer patients having the same malignant neoplasm and having been subjected to the cancer immunotherapy, wherein the data sets at least comprise expression levels of HLA-A, -B, -C, -DR isotypes from the malignant cells from each patient, and, for each patient, a score indicating patient outcome e.g. response, non-response or degree of response instigation of the cancer immunotherapy, and
z) obtaining from the neural network or other machine learning model an output, which translates into or is an indication of whether the patient has a low likelihood of responding to therapy.

In some embodiments, the cancer immunotherapy is treatment comprising or consisting of administration of immune checkpoint inhibitor(s). As shown in the examples, the per patient outcome (in terms of progression or non-progression of malignant disease) in a clinical trial of a therapeutic regimen involving anti-PD1 treatment of malignant melanoma could be predicted with very high precision when integrating the expression levels or of both MHC Class I and MHC Class II Isotypes in each individual patient's tumour microenvironment, e.g. sampled by a tumour biopsy. Interestingly, the finding applied to both the EVX-01 trial (patients with non-resectable melanoma) and the EVX-02 trial (patients with resectable melanoma); these clinical trials are described here:
EVX-01: https://clinicaltrials.gov/ct2/show/NCT03715985 and
EVX-02: https://clinicaltrials.gov/ct2/show/NCT04455503.
(the EVX-01 trial is also denoted the NeoPepVac trial).

It was found that the patients responding to therapy had much higher expression levels of MHC Class I and II than the non-responding patients. Further, when retroactively studying the integrated measure of MHC Class I as well as MHC Class II isotype expression levels was a superior predictor than those developed on the isolated Class I and Class II expression levels.

In other embodiments, the cancer immunotherapy comprises or consists of active immunization to induce specific adaptive immunity against neoepitopes and/or tumour associated antigens and/or endogenous retroviruses expressed by the malignant cells. Again, the clinical trials reported in the Example both relate to neoepitope identification and induction of specific immune response against the neoepitopes. Hence, in a particular interesting embodiment, the cancer immunotherapy comprises administration of immune checkpoint inhibitor(s) and further comprises active immunization to induce specific adaptive immunity against neoepitopes and/or tumour associated antigens and/or endogenous retroviruses expressed by the malignant cells. In this embodiment, where 2 types of cancer immunotherapy is received by the patient, it is in preferred embodiments ensured that the active immunization is instigated subsequent to initiation of administration of immune checkpoint inhibitor(s).

In the above-described embodiments where the active immunization entails induction of specific adaptive immunity, this is accomplished by administration of (poly) peptide vaccine agents, nucleic acid vaccine agents, in particular DNA or RNA vaccine agents, viral vaccine agents, or bacterial vaccine agents. These embodiments generally follow the generally known approaches in the art, and the vaccine agents, formulations, and administration regimens as well as the identification of useful vaccine agents are in particularly preferred embodiments those disclosed in international patent publications WO 2020/141207, WO 2020/182901, WO 2021/048381, WO 2021/048400, and WO 2021/123232, as well as in still unpublished European and international patent application nos.: EP 201887033.1, EP 21153781.6, PCT/EP2021/059117, EP20185743.0, and 20207526.3.

As apparent from the examples, it turns out that the expression levels of MHC Class I and II molecule isotypes by the cells of the microenvironment of the patient's malignant neoplasm are advantageously determined in at least one tumour cell-containing sample from the patient, and in particular from the at least one of the patient's lymph nodes. First of all, sampling from lymph nodes is technically more simple than sampling from tumour tissue in other locations in the body, and it further turns out that the fidelity of the method of the first aspect is superior when the tumour cell material has been obtained from lymph nodes.

The MHC Class I molecule isotypes whose expression levels are determined in step a) are selected from HLA-A, HLA-B, HLA-C, HLA-E, HLA-F, and HLA-G, and preferably are all of HLA-A, HLA-B, HLA-C, HLA-E, HLA-F, and HLA-G. However, it has turned out that the predictive value in the method of the invention is very high when no more than the HLA-A, -B, -C isotypes are determined and used in the predictions—it turns out that all of these HLA isotypes have a positive weight for determination of the likelihood of response to therapy, hence the fact that they in the method of the first aspect are all weighted with the same sign (either positive or negative).

MHC Class 11 molecule isotypes whose expression levels are determined in step a) are selected from HLA-DRA, HLA-DRB1, HLA-DPA1, HLA-DPB1, HLA-DQA1, and HLA-DQB1, and preferably are all of HLA-DRA, HLA-DRB1, HLA-DPA1, HLA-DPB1, HLA-DQA1, and HLA-DQB1. However, as is the case for the Class 1 molecules, a subset of these (HLA-DR1 and HLA-B) contribute predominantly to the overall predictive value, hence the fact that these are weighted with the same sign as HLA-A, -B, and -C.

In preferred embodiments, the cancer immunotherapy comprises administration of immune checkpoint inhibitor(s), targeting at least one immune checkpoint selected from the group consisting of CTLA-4, PD1, PDL1, LAG-3, TIM-3, B7-H3, and 87-H4. PD1 is particularly preferred. Hence the cancer immunotherapy can comprise administration of an immune checkpoint inhibitor selected from the group consisting of Ipilimumab, Cemiplimab, Pembrolizumab, Nivolumab, Atezolizumab, Avelumab, Durvalumab, Relatlimab, LAG525, REGN3767, BI 754111, FS118, Sym023, TSR-022, MGC018, and FPA150.

As mentioned above, a simple linear multivariate regression model has proven effective in enabling interpolation or extrapolation from MHC expression levels in patients' tumor microenvironments to obtain a combined expression score value, which in turn translates directly into a likelihood of responding to immunotherapy. However, it cannot be ruled out that more sophisticated means and methods for evaluating complex data could be equally or even better suited. So, in embodiments of first aspect of the invention, calculation in step b) of the combined expression score value comprises inputting the expression levels from step a) into a machine learning model such as a neural network, which has been trained/programmed with historical patient data sets comprising—for each historical patient data set—at least MHC Class I and II isotype expression levels by the cells in the patient's tumor microenvironment and a clinical outcome indicator for the patient and obtaining the combined expression score value as output from the machine learning model; or (more simply) interpolating or extrapolating the combined expression score value (or the likelihood of response to therapy) by inputting the expression levels of step a) into a multivariate regression model, which takes as independent variables historical patient data sets of MHC Class I and II isotype expression levels and as dependent variable a clinical outcome Indicator from each historical data set.

In a preferred embodiment, the multivariate regression model is established by applying LDA (linear discriminant analysis) in order to remove noise from the model (i.e. contributions from irrelevant or confounding MHC isotype expression levels) and to avoid overfitting. It is, as demonstrated in the examples, for instance possible to us the two first principal components from a principal component analysis (PCA) as input to the LDA model, where the PCA is performed on data set(s) that comprise expression level data for MHC isotypes and related clinical responses to cancer immunotherapy.

The determination in step c can be integrated with the calculation of the combined expression score value and/or wherein the determination is the result of an analysis of variance (ANOVA). Such an integration can in the first case simply rely on a threshold value (e.g. the 95% confidence level mentioned above but also on a more simple “more likely than not”-evaluation) so that the output from the method is binary: (“high likelihood of response to therapy” and “not high likelihood of response to therapy”) depending on the combined expression score value. In this connection, reference is also made to the discussion of machine learning models and neural networks in the beginning of the present section.

It is to be noted that the 95% confidence level set in step c2 is not a fixed threshold for determining whether a patient is likely to respond or not. The standard applied is rather that the test utilised must be able to determine whether it is more likely than not that a given patient will respond to the cancer immune therapy. That is, a patient of high likelihood of response to therapy will be one for whom it is more likely than not that the patient will respond to the cancer immune therapy, and a patient exhibiting a “not high likelihood of response to therapy” will be one for whom it is more likely than not that the patient will not respond to the cancer immune therapy.

However, current experience has shown that patients in the two groups generally do not overlap with respect to their combined expression score values, meaning that the selection of patients that will respond to therapy will not change to any substantial degree even if the above-mentioned 95% confidence level would be set to 97.5% or 90%.

One convenient way of assigning a likelihood for treatment success is that the determination in step c is expressed in terms of a probability of development of progressive disease within a pre-selected period of time after instigation of treatment with cancer Immunotherapy. Early progression is an indicator that the immunotherapy has no significant effect, and in such cases, the immunotherapy should be discarded with in exchange for alternative treatment regimens that can be offered to the patient.

This pre-selected period of time can vary and is to a certain extent dependent on the general characteristics of the malignant neoplasm being treated. For instance, where aggressive disease can render it fully acceptable that progression occurs at a relatively early time after treatment instigation because the treatment would nevertheless provide for prolonged survival or improved quality of life in a period after instigation of treatment. In other cases, where development of the malignant neoplasm is slow, the pre-selected period of time will be longer. In general, the pre-selected period will be chosen with a view to ensure with a high certainty that the individual patient would truly gain a benefit from the treatment. So, the pre-selected period of time is selected from 10 weeks, such as 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 weeks.

### 2^{nd} and 3^{rd} Aspects of the Invention

Since the method of the 1^{st} aspect of the invention enables the identification of those patients that will ultimately benefit from treatment with cancer immunotherapy (i.e. the responders to therapy), it also becomes possible to rationally treat patients that will benefit with a very high likelihood as well as to offer alternative therapies for those patients that have a low likelihood of benefitting (the non-responders). Aspects 2 and 3 disclosed above both relate to this utilisation of the result of the method of the first aspect.

### Features of Relevance for all Embodiments of the Aspects of the Invention

Generally, the malignant neoplasms (tumours) considered in the various aspects of the invention and their embodiments are the following, grouped on the basis of their histological origin: an epithelial tumour, a non-epithelial tumour, and a mixed tumour. The epithelial tumour may be both a carcinoma or an adenocarcinoma, and the non-epithelial tumour or mixed tumour is typically a liposarcoma, a fibrosarcoma, a chondrosarcoma, an osteosarcoma, a leiomyosarcoma, a rhabdomyosarcoma, a glioma, a neuroblastoma, a medulloblastoma, a malignant melanoma, a malignant meningioma, a neurofibrosarcoma, a leukemia, a myeloproliferative disorder, a lymphoma, a hemangiosarcoma, a Kaposi's sarcoma, a malignant teratoma, a dysgerminoma, a seminoma, or a choriosarcoma.

Also, the anatomic location of the malignant neoplasm (tumour) can be anywhere in body. So the tumour may be a of the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the ovaries, the uterus, the uterine cervix, the spleen, bone, lung, or cartilage.

Particularly Important malignant neoplasms are those where treatment with immune checkpoint inhibitors have been successful in clinical trials and preferably approved by regulatory bodies such as the FDA and EMA. A list of such malignant neoplasms is provided at https://en.wikipedia.org/wiki/Checkpoint inhibitor and currently lists the following malignant neoplasms: Basal Cell Carcinoma, Bladder Cancer, Breast Cancer, Cervical Cancer, Colorectal Cancer, Endometrial Cancer, Esophageal Carcinoma, Gastric Cancer, Head and Neck Cancer, Hepatocellular Carcinoma, Hodgkin's Lymphoma, Malignant Pleural Mesothelioma, Merkel Cell Carcinoma, Metastatic Melanoma, Non-Small Cell Lung Cancer, Renal Cell Carcinoma, Small Cell Lung Cancer, Squamous Cell Carcinoma, and Urothelial Carcinoma.

The immune checkpoint inhibitor is in principle any agent, which specifically binds to and interferes with an immune checkpoint's function as an immune modulator; as such antibodies (in particular monoclonal), antibody fragments and antibody analogues are useful (see the generic description of various antibody-related molecules in the definition section above), but also soluble receptors or small molecules that have the same inhibitory effects are of relevance.

Currently, the immune checkpoints that are targeted are selected from the group consisting of CTLA-4, PD1, PDL1, LAG-3, TIM-3, 87-H3, and B7-H4, and the most important immune checkpoint inhibitors are selected from the group consisting of Ipilimumab, Cemiplimab, Pembrolizumab, Nivolumab, Atezolizumab, Avelumab, Durvalumab, Relatlimab, Relatlimab, LAG525, REGN3767, BI 754111, FS118, Sym023, TSR-022, MGC018, and FPA150.

The present invention is based on findings from 2 clinical studies on patients with two different stages of malignant melanoma, respectively, where the patients received treatment with the checkpoint inhibitor targeting PD1. It is hence preferred in all aspects and embodiments disclosed herein that the malignant neoplasm is malignant melanoma and that the treatment comprises administration of a checkpoint inhibitor targeting PD1 (e.g. the checkpoint inhibitors Nivolumab, Pembrolizumab, and Cemiplimab).

### Determination of Expression Levels the Malignant Neoplasm Microenvironment

The expression levels can be determined by any method known in the art: RNA-sequencing, such as poly-A enriched or total RNA bulk sequencing, Ribo-sequencing and single cell sequencing technologies; mass spectrometry, in particular quantitative MS technologies; cell sorting, such as fluorescence assisted cell sorting (FACS); immunohistochemical techniques; microarray techniques; quantitative PCR (qPCR), including RT-PCR; Western blotting and Northern blotting.

### Example 1

### Identification of Prognostic Markers

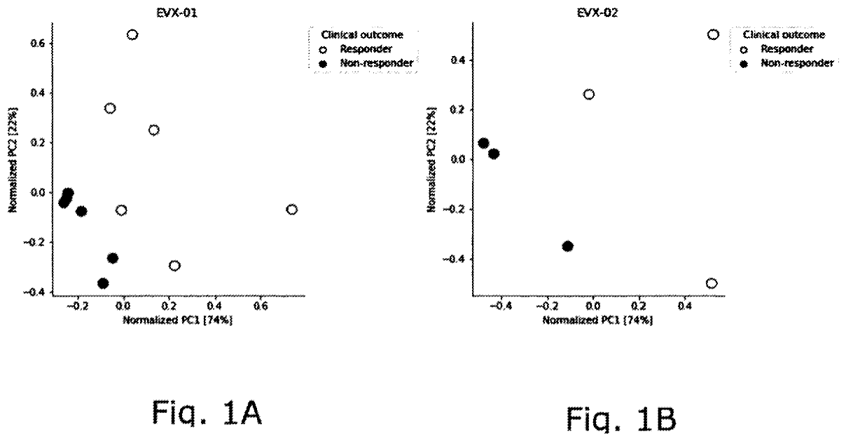

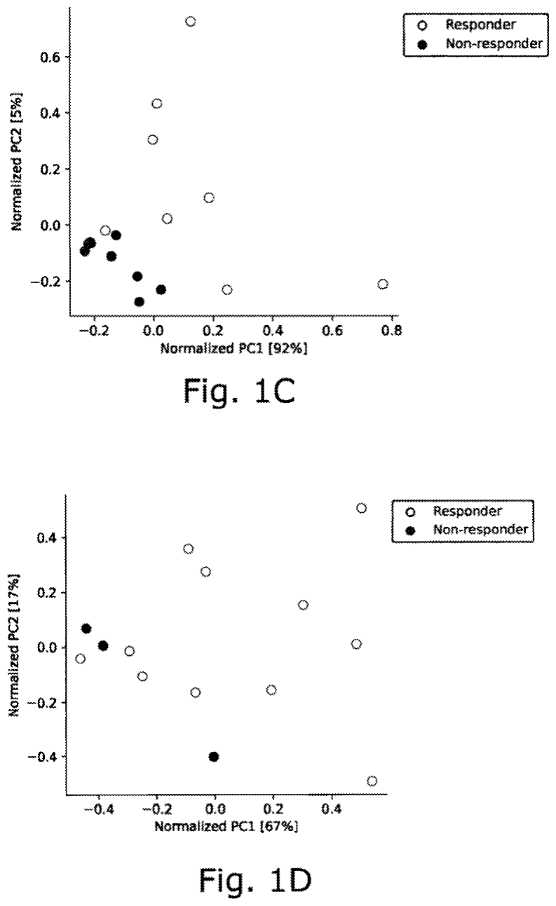

Tumor biopsies were collected from patients when they enrolled in the EVX-01 and the EVX-02 trial (neoepitope treatment in combination with anti-PD1, respectively with neoepitope delivery in the form of peptides and DNA plasmids). Biomarker expression levels were quantified through RNA sequencing of the tumor biopsy (mapping to reference genome with STAR and quantification with RSEM). Principal component analysis (PCA) shows that patients largely are clustered based on response to therapy, see FIG. 1A and FIG. 1B from the EVX-01 and EVX-02 trials respectively.

The EVX-01 trial was employed as an example for model generation using Linear Discriminant Analysis (LDA). The two first principal components from the PCA were used as input to the LDA model for noise reduction as well as to reduce the number of variables and avoid overfitting.

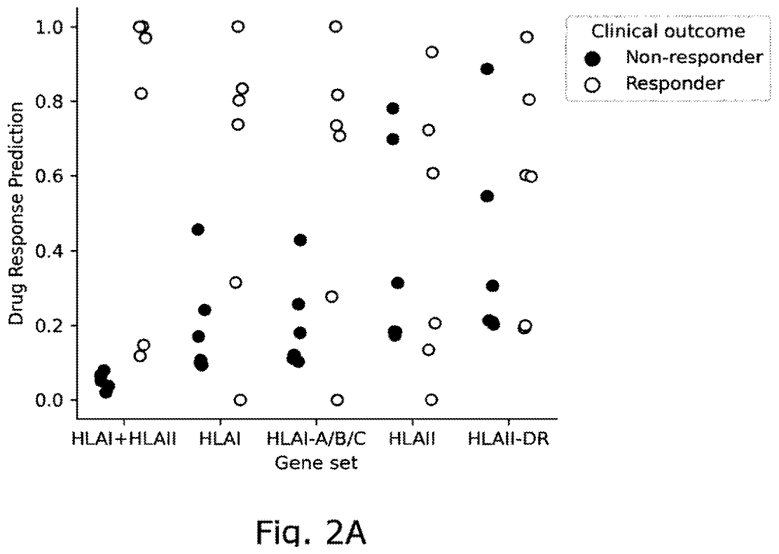

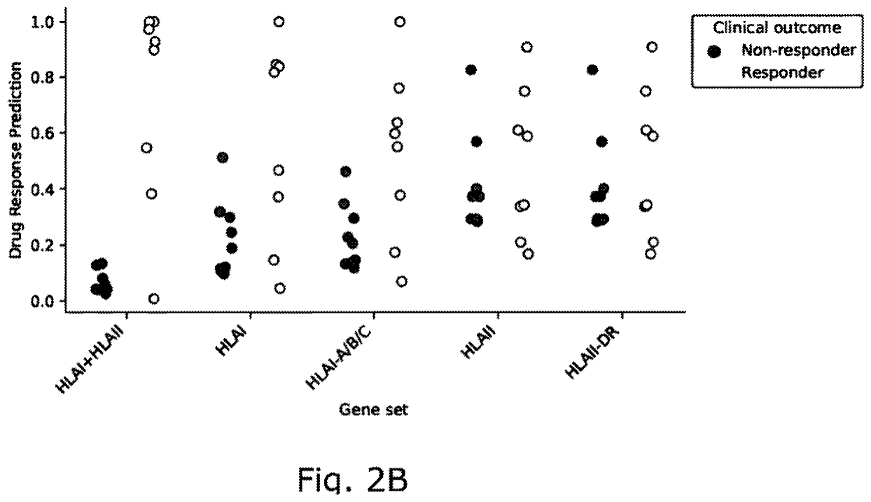

A leave-one-patient-out cross validation study was conducted to demonstrate the predictive performance of the model. A comparison of the model based on PCA derived from the full gene set (HLA-A/B/C/E/F/G/DRA/DRB1/DPA1/DPB1/DQA1/DQB1) was compared to models based on PCAs derived from the respective gene sets (HLAI classical: HLA-A/B/C), (HLA-1: HLA-A/B/C/E/F/G), (HLA-II: HLA-DRA/DRB1/DPA1/DPB1/DQA1/DQB1) and (HLAII-DR: HLA-DRA/HLA-DRB1). The model based on the full gene set (HLAI+HLAII) is superior in segregating the data, cf. FIG. 2.

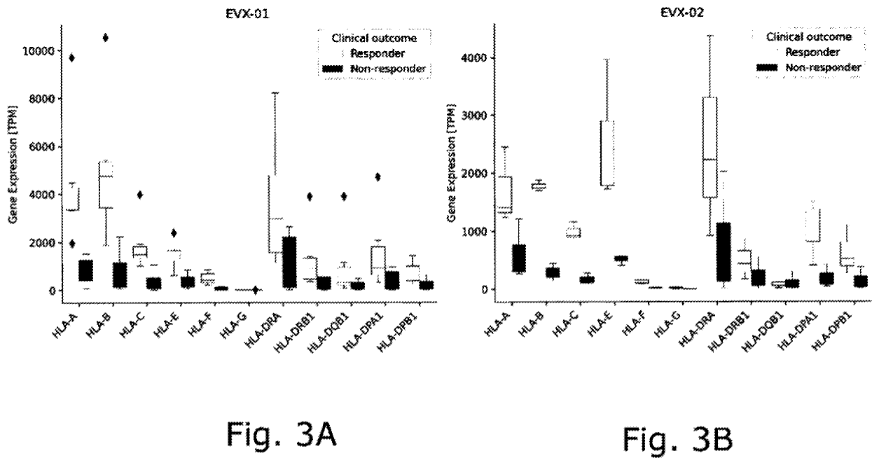

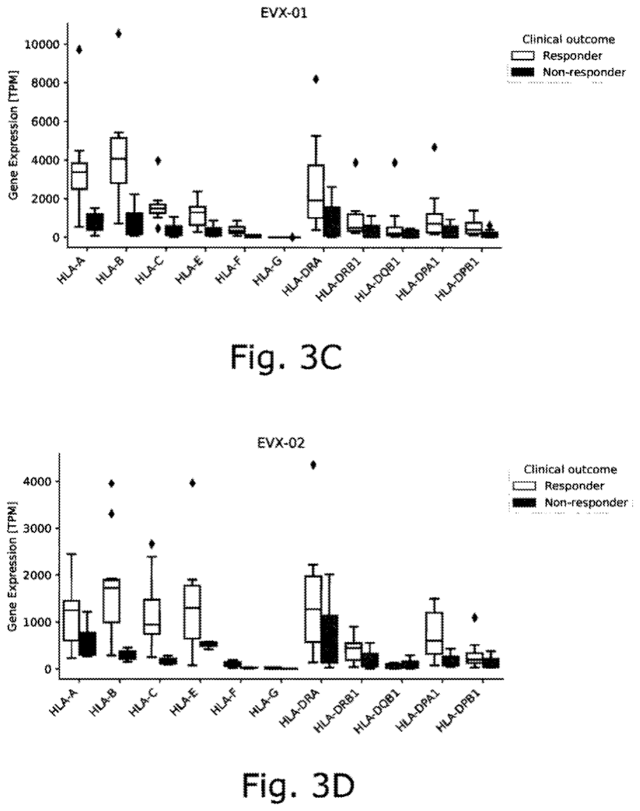

Finally, it was in both the EVX-01 and EVX-02 trial investigated how responses to treatment correlated with expression of each of the HLA isotypes tested. As can be seen from FIG. 3A (EVX-01 trial) and FIG. 3B (EVX-02 trial), a positive response to treatment in all cases correlated positively with the expression levels.

### Example 2

### Further Data on Prognostic Markers

Continued enrolment in the clinical trials EVX-01 and EVX-02 since the data presented in Example 1 became available has increased the cohort sizes on which to base the analysis; from 12 to 18 patients in the EVX-01 trial and from 6 to 14 patients in the EVX-02 trial.

A very strong separation continues to be observed between patients based on whether they respond to therapy (FIG. 1C) In EVX-01, while a trend can be observed in EVX-02 (FIG. 1D) but harder to establish given the few cases of patients relapsing into progressive disease.

The leave one-patient out analysis demonstrates that in particular predictions based on HLA class I genes are able to stratify patient outcomes, yet a combined model of HLA class 1 and HLA class II improves the patient stratification even further (see FIG. 28).

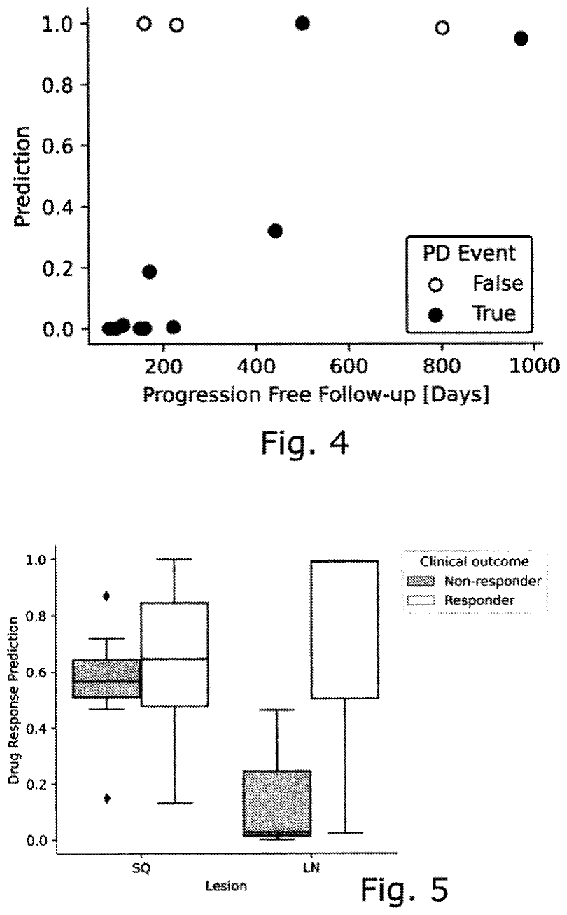

The predictions can also be viewed through the lens of progression free survival, i.e. the time until a patient has a progressive disease event or in case of no event the follow-up time. Follow-up time was not provided for all patients in the trials, thus this analysis only comprises the subset of the patients, for whom follow-up data are available. From FIG. 4, it is evident that patients that experience disease progression are predicted with a low value, while those with a medium prediction value have an intermediate disease-free duration and arguably benefit to some degree from the treatment. Finally, those with high prediction either have a long disease-free period or have not experienced any disease progression.

Finally, we show that in both EVX-01 and EVX-02, the responding patients have a higher expression level of the HLA genes compared to those that do not respond, see FIG. 3C and FIG. 3D.

As a final measure, a publicly available study of advanced melanoma (stage III/IV) treated with anti-PD1 (pembro or nivo) was subjected to further analysis. RNA-seq profiles of baseline biopsies prior to treatment were used for quantifying the HLA expression levels and models developed using the leave-one-patient out approach. While the sample size in this study is limited, we observed that lymph node biopsies appear more predictive of the clinical outcome as compared to skin biopsies, see FIG. 5.

1. A method for determining the likelihood of a human patient suffering from a malignant neoplasm to respond to a later received cancer immunotherapy, the method comprising,
a) quantitively determining the expression levels of MHC Class I and II molecule isotypes by the cells of the microenvironment of the patient's malignant neoplasm,
b) calculating a combined expression score value from weighted expression levels of at least HLA-A, B, -C, and -DR isotypes determined in step a, wherein the weights for all of isotypes HLA-A, B, C, and -DR have the same sign (either positive or negative), and
c) determining that the human patient has a low likelihood of responding to therapy if the calculated combined expression score value from step b is either
1) closer to the combined expression score values determined in other patients suffering from the same malignant neoplasm and not responding to said cancer immunotherapy than to the combined expression score values from other patients suffering from the same malignant neoplasm and having exhibited response to said cancer immunotherapy, and/or
2) not significantly different at a 95% confidence level from the combined expression score values obtained from other patients suffering from the same malignant neoplasm and having exhibited lack of response to said cancer immunotherapy.

2. The method according to claim 1, wherein said cancer immunotherapy is treatment comprising or consisting of administration of immune checkpoint inhibitor(s).

3. The method according to claim 1, wherein said cancer immunotherapy comprises or consists of active immunization to induce specific adaptive immunity against neoepitopes and/or tumour associated antigens and/or endogenous retroviruses expressed by the malignant cells.

4. The method according to claim 1, wherein said cancer immunotherapy comprises administration of immune checkpoint inhibitor(s) and further comprises active immunization to induce specific adaptive immunity against neoepitopes and/or tumour associated antigens and/or endogenous retroviruses expressed by the malignant cells.

5. The method according to claim 4, wherein the active immunization is instigated subsequent to initiation of administration of immune checkpoint inhibitor(s).

6. The method according to claim 3 wherein the active immunization entails administration of (poly) peptide vaccine agents, nucleic acid vaccine agents, in particular DNA or RNA vaccine agents, viral vaccine agents, and bacterial vaccine agents.

7. The method according to claim 1, wherein the expression levels of MHC Class I and II molecule isotypes by the cells of the microenvironment of the patient's malignant neoplasm are determined in at least one tumour cell-containing sample from the patient.

8. The method according to claim 7, wherein the at least one tumour cell-containing sample is obtained from at least one of the patient's lymph nodes.

9. The method according to claim 1, wherein the MHC Class I molecule isotypes whose expression levels are determined in step a) are selected from HLA-A, HLA-B, HLA-C, HLA-B, HLA-F, and HLA-G, and preferably are all of HLA-A, HLA-B, HLA-C, HLA-E, HLA-F, and HLA-G.

10. The method according to claim 1, wherein the MHC Class II molecule isotypes whose expression levels are determined in step a) are HLA-DRA, HLA-DRB1, HLA-DPA1, HLA-DPB1, HLA-DQA1, and HLA-DQB1, and preferably are all of HLA-DRA, HLA-DRB1, HLA-DPA1, HLA-DPB1, HLA-DQA1, and HLA-DQB1.

11. The method according to claim 1, wherein the cancer immunotherapy comprises administration of immune checkpoint inhibitor(s) targeting at least one immune checkpoint selected from the group consisting of CTLA-4, PD1, PDL1, LAG-3, TIM-3, B7, H3, and B7-H4.

12. The method according to claim 1, wherein the cancer immunotherapy comprises administration of an immune checkpoint inhibitor selected from the group consisting of Ipilimumab, Cemiplimab, Pembrolizumab, Nivolumab, Atezolizumab, Avelumab, Durvalumab, Relatlimab, LAG525, REGN3767, BI 754111, FS118, Sym023, TSR-022, MGC018, and FPA150.

13. The method according to claim 1, wherein the calculation in step b is based on Linear Discriminant Analysis (LDA) to arrive at the weights of the same sign.

14. The method according to claim 13, wherein the LDA is fed with components 1 and 2 of a principal component analysis of data set(s) comprising MHC isotype expression levels and responses to cancer immunotherapy.

15. The method according to claim 1, wherein calculation in step b) of the combined expression score value comprises
i. inputting the expression levels from step a) into a machine learning model such as a neural network, which has been trained/programmed with historical patient data sets comprising for each historical patient data set at least MHC Class I and II isotype expression levels by the patient's tumour microenvironment and a disease progression indicator for the patient and obtaining the combined expression score value as output from the machine learning model; or
ii. interpolating or extrapolating the combined expression score value by inputting the expression levels of step a) into a multivariate regression or classification model, which takes as independent variables historical patient data sets of MHC Class I and II isotype expression levels and as dependent variable a clinical outcome indicator from each historical data set.

16. The method according to claim 1, wherein the determination in step c is expressed in terms of a probability of development of progressive disease within a pre-selected period of time after instigation of treatment with cancer immunotherapy.

17. The method according to claim 16, wherein the pre-selected period of time is selected from 10 weeks, such as 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 weeks.

18. The method according to claim 1, wherein the determination in step c is integrated with the calculation of the combined expression score value and/or wherein the determination is the result of an analysis of variance (ANOVA).

19. A method for treatment of a human patient suffering from a malignant neoplasm, comprising determining by the method according to claim 1, whether the human patient has low likelihood of responding to said cancer immunotherapy, and subsequently subjecting the human patient to the cancer immunotherapy if it is determined that the likelihood of responding is different from low, and subjecting the patient to palliative or alternative treatment regimens if it is determined that the likelihood of responding is low.

20. A method for determining whether a human patient suffering from a malignant neoplasm is eligible for a cancer immunotherapy, the method comprising determining by the method according to claim 1, whether the human patient has a low likelihood of responding to said cancer immunotherapy and concluding that the patient is eligible for said cancer immunotherapy if the determination reveals that the patient does not have a low likelihood of responding to therapy.

21. The method according to claim 19 or 20, wherein the cancer immunotherapy is selected from the group consisting of
a) a treatment comprising or consisting of administration of immune checkpoint inhibitor(s),
b) a treatment comprising or consisting of active immunization to induce specific adaptive immunity against neoepitopes and/or tumour associated antigens and/or endogenous retroviruses expressed by the malignant cells, and
c) a treatment comprising administration of immune checkpoint inhibitor(s) and further comprising active immunization to induce specific adaptive immunity against neoepitopes and/or tumour associated antigens and/or endogenous retroviruses expressed by the malignant cells.

22. The method according to claim 1, wherein the malignant neoplasm is selected from an epithelial tumour, a non-epithelial tumour, and a mixed tumour.

23. The method according to claim 22, wherein the epithelial tumour is a carcinoma or an adenocarcinoma, and the non-epithelial tumour or mixed tumour is a liposarcoma, a fibrosarcoma, a chondrosarcoma, an osteosarcoma, a leiomyosarcoma, a rhabdomyosarcoma, a glioma, a neuroblastoma, a medulloblastoma, a malignant melanoma, a malignant meningioma, a neurofibrosarcoma, a leukemia, a myeloproliferative disorder, a lymphoma, a hemangiosarcoma, a Kaposi's sarcoma, a malignant teratoma, a dysgerminoma, a seminoma, or a choriosarcoma.

24. The method according to claim 1, 19 or 20 wherein the malignant neoplasm is a tumor of the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the ovaries, the uterus, the uterine cervix, the spleen, bone, lung, or cartilage.

25. The method according to claim 1, 19, or 20, wherein the malignant neoplasm is selected from the group consisting of, Basal Cell Carcinoma, Bladder Cancer, Breast Cancer, Cervical Cancer, Colorectal Cancer, Endometrial Cancer, Esophageal Carcinoma, Gastric Cancer, Head and Neck Cancer, Hepatocellular Carcinoma, Hodgkin's Lymphoma, Malignant Pleural Mesothelioma, Merkel Cell Carcinoma, Metastatic Melanoma, Non-Small Cell Lung Cancer, Renal Cell Carcinoma, Small Cell Lung Cancer, Squamous Cell Carcinoma, and Urothelial Carcinoma.
